(19) United States
(12) Patent Application Publication
(10) Pub. No.: US 2003/0120128 A1
(43) Pub. Date: Jun. 26, 2003

(54) Low-energy brachytherapy sources

(76) Inventors: Richard Taschereau, Sainte-Foy (CA); Jean Pouliot, Mill Valley, CA (US); Rene Roy, Sainte-Foy (CA)

Correspondence Address: Mr. Marc Lupien, c/o Goudreau Gage Dubuc, Stock Exchange Tower, 800 Place Victoria, Suite 3400, Montreal, QC, H4Z 1E9, CA

(21) Appl. No.: 10/261,759

(22) Filed: Oct. 2, 2002

Related U.S. Application Data
(60) Provisional application No. 60/326,734, filed on Oct. 4, 2001.

(51) Int. Cl. A61N 5/00

(52) U.S. Cl. 600/1

(57) ABSTRACT

A radioactive implant characterized by a biological effectiveness comparable to that of palladium-103 and, simultaneously, by a half-life of the order of that of iodine-125. More specifically, the present invention provides a radioactive implant having improved biological effectiveness, by lowering the average energy of the photon spectrum of a relatively long half-life radioisotope element using x-ray fluorescence. Selection of a fluorescent material within elements having an atomic number between 39 and 45, preferably between 40 and 42, for surrounding a radioactive inner core comprising the radioisotope element, is described.

### FIELD OF THE INVENTION

The present invention relates to a low-energy brachytherapy source. More specifically, the present invention is concerned with a brachytherapy source that combines the advantages of a long half-life and a high biological effectiveness.

### BACKGROUND OF THE INVENTION

Ever since the discovery of X-ray radiation and its effects on living tissues, in the end of the 19^{th} century, methods for using this type of radiation and its ability to kill cells have been developed and used in the treatment of tumors.

External beam therapy is a first method for treating tumors, whereby high-energy radiation beams are directed to the patient's body from the outside. A drawback of such a method is that the high-energy radiation beams may encounter healthy tissue before reaching the tumor itself, therefore inducing damages to tissues that are not part of the tumor.

Various improvements minimize this problem, consisting, for example, in rotating the X-ray source around the patient so as to maximize the focus of the radiation at the site of the tumor.

Brachytherapy stands as an alternative to external beam therapy. The term brachytherapy means “short-range therapy”, from the Greek word “brachys”: short, close. It generally uses a source of radiation physically located directly inside the tumor, or very close to the tumor. The source of radiation may be temporarily or permanently placed inside the patient's body.

By selecting carefully chosen radioisotope elements according to both their half-life and their radiation energy spectrum (considering the energy and the type of the radiation desired), it is possible to irradiate the tumor while keeping the effects on the surrounding healthy tissue minimal. In the event that the half-life of the radioisotope element is sufficiently short, and provided the material encapsulating the radioactive source is biocompatible, it may be left inside the patient's body after the brachytherapy treatment ends.

The idea of brachytherapy has been known for a long time, as testified, for example, by U.S. Pat. No. 1,494,861 issued in 1924 to Viol for his “Radium applicator”. The use of radium as the radioisotope element means using a very high-energy radiation, which can be harmful to the people attending the patient being treated. Additionally, because of the long half-life of radium (close to 1600 years), such applicators must be removed from the patient's body at the end of the treatment.

Thereafter, more convenient and efficient brachytherapy sources were developed. In 1967, Lawrence, in U.S. Pat. No. 3,351,049, describes a radioactive therapeutic seed made of radioisotope elements such as iodine-125, palladium-103 and cesium-131. Such radioisotope elements having a short half-life, the source gradually loses its activity at the end of the treatment period, and therefore it can be left in the patient's body. Moreover, the use of these radioisotope elements poses no threat to surrounding tissues or to other persons because of their lower energy.

Furthermore, Lawrence suggests materials that should preferably be used for making the outer shell encapsulating the radioactive seed, taking into account various parameters, such as the X-ray absorption cross-section by the outer shell and the high strength, corrosion resistance and biocompatibility required for this outer shell. Titanium and stainless steel are particularly well suited for this application. An outer shell is not an essential feature of a brachytherapy source. Its presence is desired when it constitutes a permanent implant or when its residence time in a recipient's body is long. A source having no outer shell however can be inserted as is or in a tube like a catheter, for insertion in a tissue such as a blood vessel, during an angioplasty, for example, which would result in prevention of restenosis.

Finally, Lawrence's patent also teaches using an element of high atomic number inside the outer shell. This element should be carefully located away, as much as possible, from the path of the radiation between the core and the outer shell of the brachytherapy source. A high atomic number element being opaque to X-ray radiation, it enables the visualization of the seeds for the purposes of localization and counting by standard x-ray photographic techniques.

Generally stated, brachytherapy sources used for cancer treatment comprise a radioactive inner core, made of a radioisotope element emitting photon radiation within the energy range comprised between 15 keV and 600 keV. The radioisotope is usually immobilized on a solid support, sealed inside a tiny biocompatible encapsulating shell. The preferred radioisotope elements used in the radioactive inner core are usually iodine-125 or palladium-103 for low-dose treatments, and iridium-192 for high-dose treatments, whereas a preferred metallic material for the outer shell is titanium. Silver and gold are commonly used as radio-opaque materials in permanent implants. An appropriate number of such sources are inserted inside a tumor so that the tumor is destroyed by the emitted radiation.

As an alternative, in U.S. Pat. No. 4,323,055 issued to Kubiatowicz in 1982, use is made of a silver rod both as the carrier of the iodine in the radioactive inner core and as the radio-opaque element, thus simplifying the manufacturing process.

A problem sometimes addressed in the art concerns the anisotropy of the angular distribution of the radiation. This anisotropy results in a non-uniform radiation emitted by the seed, which may cause “cold spots”, i.e. regions of the tumor that receive less radiation. U.S. Pat. No. 6,099,458 addresses this problem by designing a seed characterized by an improved isotropy.

One major drawback of palladium-103 for making the seed is its short half-life (17 days). U.S. Pat. No. 4,702,228, issued to Russell in 1987, teaches using a palladium that is significantly enriched in palladium-102 and exposing it to neutron flux for activation, preferably after the encapsulation.

As can be seen from the above discussion, significant efforts have been invested in the design of improved radioactive seeds, and the ever-increasing understanding and knowledge of their therapeutic capabilities in the medical field have made the use of radioactive seed therapy a well accepted medical procedure for the treatment of diseased tissues.

As of today, iodine-125 and palladium-103 are by far the preferred radioactive materials for the manufacture of therapeutic seeds. However, both materials have their own merits and disadvantages. In particular, palladium-103 has a more favorable X-ray spectrum than iodine-125, whereas iodine-125 has a longer half-life (60 days compared to 17 days for palladium-103). These are precisely the two main parameters that determine the effectiveness of a radiotherapeutic treatment, since they define the duration of the treatment and the biological effectiveness of the radiation.

More specifically, in the context of brachytherapy, biological effectiveness relates to the capacity of a radiation to kill cancerous cells. It is a well-known fact in the art that high energies provide longer penetration paths in living tissues (see ICRU Report 30—“Quantitative Concepts and Dosimetry in Radiobiology”, International commission on radiation units and measurement, Washington D.C., 1979), including tumors, whereas low energies are more effective in causing death of target cells in close proximity (see “ICRU Report 36—Microdosimetry”, International commission on radiation units and measurement, Bethesda, Md., 1983; Wuu, C. S. and Zaider, M., A Calculation of the relative biological effectiveness of 1251 and 103Pd brachytherapy sources using the concept of proximity function, Med. Phys. 25 (11), 1998, 2186-89; Ling CC, Li WX, Anderson LL. The relative biological effectiveness of 1-125 and Pd-103. Int. J. Radiat. Oncol. Biol. Phys. May 15, 1995; 32(2):373-8; and Hall, Eric J., Radiobiology for the radiologist, Philadelphia, J. B. Lippincot Company, 1994, 478 p).

Because of the shorter penetration paths of lower energy radiation, an increase in the effectiveness of a radiation so as to cause death of the target cells is achieved at the expense of a shorter penetration path. Therefore, a balance needs to be achieved between these two conflicting parameters. Such a balance is reasonably well achieved with energies in the vicinity of 20 keV.

From the sole point of view of its intrinsic radiation spectrum, palladium is considered to be very close to optimal, since this radioisotope element combines a good penetration in the tumor and a high killing effectiveness. However, its relatively short (17 days) half-life is not quite adequate in the treatment of slow growing tumors such as prostate tumors, for example.

Efforts have been made recently towards more efficient implants. One method relies on modifying the photon energy spectrum of iodine-125 by using X-ray fluorescence. As is a common knowledge, X-ray fluorescence refers to the emission of an X-ray photon by an excited atom after that atom has been ionized. While most materials exhibit X-ray fluorescence when exposed to radiation, the energy of the emitted X-rays is characteristic of the material. Moreover, it was soon found out that the most efficient X-rays on a biological point of view are those having an energy close to 20 keV. Given this requirement, only a limited number of elements are of interest in the perspective of using X-ray fluorescence for improving the photon energy spectrum of implants.

In particular, Bambinek et al., in their publication entitled: Fluorescence iodine-125 eye applicator (Med. Phys. 26 (11): 2476; 1999), disclose an eye applicator wherein iodine-125 seeds are fixed on a support having a concave shape. A fluorescent foil or layer may be inserted between the seed and the surrounding tissues. The fluorescent foil may be made of molybdenum, zirconium or rubidium, which would decrease the photon energy of the applicator down to a desired range around 20 keV. This range of values is taken from previous publications, including that by Hubbel, J. H. (Photon mass attenuation and energy absorption coefficients from 1 keV to 20 meV, in Int. J. Appl. Radiat. Isot. 33: 1269; 1982). For an optimal spatial distribution of dose for such geometry, molybdenum and zirconium were preferred.

This latter reference is primarily concerned with the spatial dose distribution of an implant having a very specific shape. There is no suggestion for any other implant shape, nor suggestions of seeking for an implant combining an energy equivalent to the energy of palladium-103 and the half-life of iodine-125. There is no suggestion either of designing a radioactive inner core that would be surrounded with a fluorescent foil, or layer, on a substantial portion of its “active” surface, so that a maximal proportion of the primary energy due to iodine-125 is shifted to a lower energy upon crossing the fluorescent foil. An “active” surface is here intended to mean a surface from which radiation is expected to be emitted so as to reach the surrounding tissues. A radiation, coming from, or crossing, this surface, is captured in the fluorescent layer, converted and transmitted as a low-energy radiation.

Therefore, in some instances such as prostate cancer where the tumors grow slowly, it is believed that there is still a need for an improved radiotherapeutic treatment. A desirable radioactive source would advantageously provide simultaneously an X-ray spectrum such as that of palladium-103 together with a longer half-life of the order of that of iodine-125, for example.

### OBJECTS OF THE INVENTION

An object of the present invention is therefore to provide an improved low-energy brachytherapy source having a suitable half-life.

### SUMMARY OF THE INVENTION

More specifically, in accordance with the present invention, there is provided an implant wherein the energy of a radioactive element having a desired half-life is modulated so as to reach an energy effective for treating tumors.

There is provided a radioactive source capable of radiating energy in a tissue wherein it is to be implanted, comprising: a radioactive inner core having an outer surface; and a layer of fluorescent material disposed around the outer surface; wherein the central radioactive inner core comprises at least one radioisotope element and the layer of fluorescent material is made of at least one sub-layer of fluorescent material that has a X-ray energy lower than the energy of the radioisotope element, so that a substantial portion, if not a major portion, of the energy of the radioisotope element is shifted within a range that is effective for preventing cell proliferation of said tissue.

It is a further object to provide a radioactive source capable of radiating energy, comprising a radioactive central, substantially cylindrical core, comprising iodine-125 and having an outer surface; and a layer of fluorescent material comprising at least one element selected from niobium, zirconium and molybdenum, and disposed around the outer surface.

There is also provided a method for fabricating a radioactive source to be implanted in a recipient's tissue, which combines the half-life of a first radioactive element and the energy spectrum of a second radioactive element that has a shorter half-life, comprising the step of shifting down the energy spectrum of the first element towards the energy spectrum of the second radioactive element by placing at least one layer of fluorescent material between the first radioactive element and the tissue to be irradiated.

Other objects, advantages and features of the present invention will become more apparent upon reading of the following non-restrictive description of preferred embodiments thereof, given by way of example only with reference to the accompanying drawings.

### BRIEF DESCRIPTION OF THE DRAWINGS

In the appended drawings:

FIG. 1 is a schematic representation of a Plexiglas™ disk used to hold a radioactive seed;

FIG. 2 shows the spectra of an unwrapped radioactive seed and of a molybdenum-wrapped radioactive seed;

FIG. 3 is a graph of the photoelectric cross-section of molybdenum.

FIG. 4 is a cross-sectional view of an implant according to a first specific embodiment of the prior art;

FIG. 5 is a cross-sectional view of an implant according to a second specific embodiment of the prior art; and

FIG. 6 is a cross-sectional view of an implant according to a specific embodiment of the present invention, which is an improved version of the implant of FIG. 5.

### DESCRIPTION OF THE PREFERRED EMBODIMENT

Generally stated, the present invention provides a brachytherapy source characterized by a biological effectiveness of one radioactive element combined to the half-life of another radioactive element.

More specifically, the present invention provides a radioactive implant having an improved biological effectiveness, by lowering the average energy of the photon spectrum of a relatively long half-life radioisotope element, taking advantage of X-ray fluorescence.

Even more precisely, the present invention provides a brachytherapy source characterized by an X-ray spectrum comparable to that of palladium-103 in the range of energy situated around 20 keV, and, simultaneously, by a half-life of the order of that of iodine-125.

To achieve such an improved radioactive implant, the radioisotope element forming the radioactive source, or substrate carrying the radioisotope element, is essentially wrapped into at least one thin layer of metal, or alloy of metals, having a lower atomic number than the radioisotope element.

In accordance with a general aspect of the present invention, there is provided a brachytherapy source comprising a radioactive inner core, with the adjunction of a fluorescent foil on at least one region of its outer surface. The radioactive inner core is made of at least one photon-emitting radioisotope element. Alternatively, the radioactive inner core may be made of a substrate carrying such photon-emitting radioisotope element. The radioactive inner core may be of a linear shape, of a circular shape or of any other suitable shape. The inner core may also be made of more than one part, or provided with an open structure. It may be opened in its center or inner surface. It may even have a linear primary shape, which is forced to take a different secondary structure. For example, a thread may become a spring-like structure.

Should the brachytherapy source be intended to be located in direct contact with human tissues, an outer shell, or capsule, made of a biocompatible material, is preferably provided for encapsulating the radioactive inner core covered with the fluorescent layer, as will be described hereinbelow. This outer shell does not need to have the same general morphology as the radioactive inner core. The fluorescent foil may also have a morphology that is substantially the same as at least a portion of the one of the inner core, or else, of the outer shell.

In accordance with an important aspect of the present invention, the fluorescent material provided for surrounding the radioactive inner core is a metal having a lower atomic number than that of the radioisotope element comprised in the radioactive inner core, and therefore a lower photon energy.

The fluorescent material can be incorporated in the brachytherapy source in a variety of ways, among which the following: in the form of a standalone metal shape surrounding the radioactive material; in the form of a thin film located either on the outer surface of the radioisotope surface (or radioisotope substrate surface) or on the inner surface of the outer layer capsule; by applying the radioisotope element inside a shape made of the fluorescent material.

The methods for applying a thin film or foil of the fluorescent material to the radioisotope element or radioisotope substrate include, in a non-restrictive sense: use of a binder phase containing very fine suspended particles of the metal or alloy of metals; physical vapor deposition; chemical vapor deposition; sputtering and other techniques known to those skilled in the art.

The methods for applying the radioisotope element inside a shape of the fluorescent material include, in a non-restrictive sense: by chemical methods, whether directly on the fluorescent material or on the silver coated fluorescent material in the case iodine is used as the radioisotope element; by electroplating; by adsorption on a fluorescent shape whose inner surface is previously treated with a binder phase containing a radioisotope immobilizing element material such as carbon, activated carbon, charcoal or any other material that will absorb radioactivity-containing fluids. Such material absorbs the liquid, so that the radioisotope gets trapped when the liquid evaporates.

The fluorescent material may alternatively be a combination of metals having lower atomic numbers than that of the radioisotope element.

Although other shapes are possible, in accordance with a particular embodiment of the present invention, a brachytherapy source is essentially cylindrical in shape, and comprises: a radioactive inner core made of iodine-125, as the radioisotope element, supported on a substrate consisting in a resin, a zeolite, or silver rod; an outer biocompatible shell made of titanium; a fluorescent layer made of a foil of molybdenum, inserted between the radioactive inner core and the outer shell.

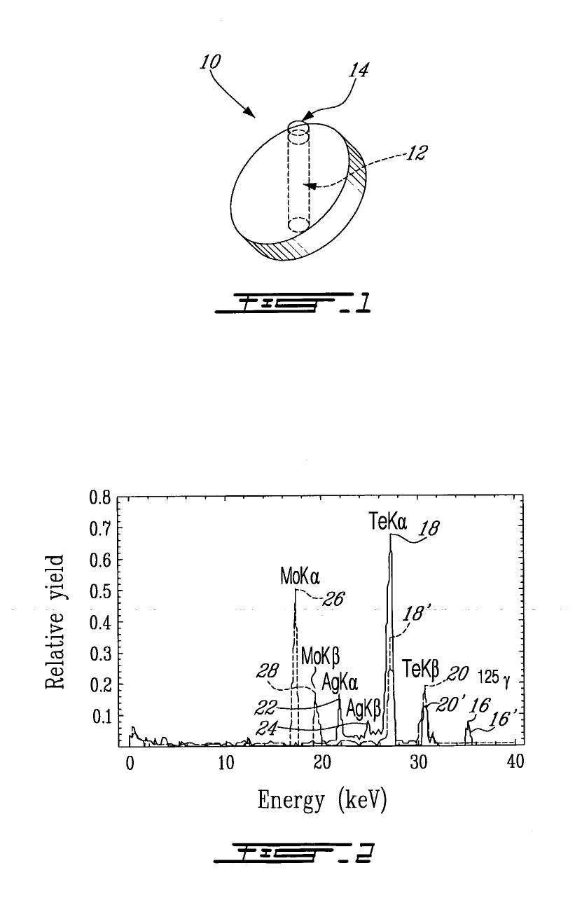

Before describing preferred embodiments of brachytherapy sources fabricated along the lines of the present invention, we will present the results of an experiment demonstrating the effect of a thin foil of molybdenum on the photon spectrum of iodine-125, with reference to FIGS. 1 to 3.

In this experiment, two commercial 0.1 milliCurie (0.1 mCi) 125-iodine seeds model LS-1 made by Draxlmage (Kirkland, Canada) are used. Each one is dropped in a channel made in a small Plexiglas disk. As is best shown in FIG. 1, such a disk 10 is 1 cm thick, has a diameter of 2 cm, and is provided with a 0.4 cm wide and 1.5 cm long channel 12.

In a first such disk, a 50 μm thick foil of molybdenum (1.4×1.4 cm) is rolled and inserted into the channel 12 prior to depositing a seed. In a second such disk, a seed is deposited by itself. For both disks, the open end 14 of the channel 12 is sealed with silicone once the seed is inserted, so as to prevent the seed from falling. Then the disks are glued at the bottom of a closeable lead container.

Spectra measurements are performed with a LINK spectrometer model XR-200. This spectrometer essentially comprises an X-ray tube and a Si/Li detector with a beryllium window. The spectrometer is provided with 1000 channels in the energy range comprised between 0 keV and 40 keV (0.04 keV per channel). It is calibrated by using the internal X-ray tube and samples of silver, rhodium and tin.

The photon energy spectrum of the seeds is measured with only the detector on (X-ray tube off). The seed with no molybdenum foil is measured first, by placing the lead container, with the cap thereof removed, at a distance of about 50 cm from the detector. About 6000 counts are collected in 500 seconds. The seed with a molybdenum foil is then measured. It has to be placed at a closer distance from the detector, due to the attenuation caused by the molybdenum foil. As an additional precaution to prevent the seed from falling inside the spectrometer, a 5 μm thick film of polycarbonate is taped in front of the lead container. The polycarbonate film, which was not used with the first measurement, is specially designed not to interfere with X-ray measurements. It is made of a low-Z material and very thin. About 15000 counts are collected in 500 seconds.

In FIG. 2 are shown the normalized spectra obtained from both seeds, i.e. relative yield versus energy in keV. The normalization is such that the area under the curve is equal to 1. The full line corresponds to the spectrum of the LS-1 seed without molybdenum foil. This spectrum exhibits well-defined features: a gamma (label 16) at 35.5 keV and two X-rays, labeled 18 and 20 respectively, due to tellurium, at 27.4 keV and 31 keV successively. Additional X-rays can also been seen at 22.1 keV (label 22) and 24.9 keV (label 24); they originate from silver, the seed containing two beads with a small amount of silver.

For comparison purposes, the dashed line represents the spectrum of the LS-1 seed wrapped in a molybdenum foil. This second spectrum displays the same well-defined peaks, respectively 16′, 18′, and 20′. It is to be noted however that the Te K_{α} peak (label 18′) is reduced by about 50%, while no silver peak is to be seen. Two additional features appear at 17.4 keV (label 26) and 19.6 keV (label 28), which are characteristic X-rays due to molybdenum. The absence of the silver peak and the reduction of the Te K_{α} peak (label 18′) are explained by the absorption of the corresponding photons by the molybdenum foil.

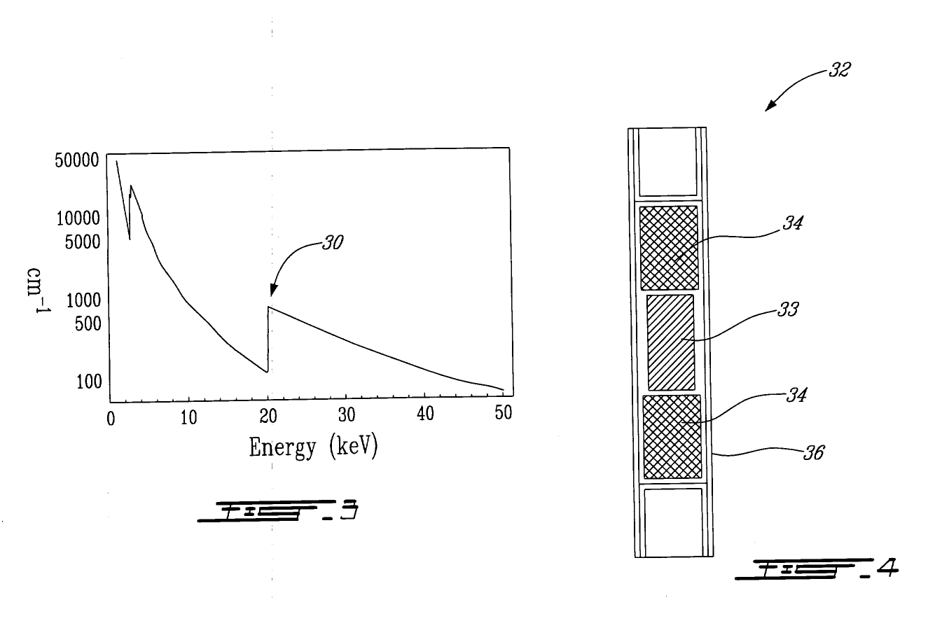

Indeed, as displayed in FIG. 3, molybdenum shows a maximum photon absorption at 20 keV, which corresponds to the so-called K-edge (label 30). Since X-rays due to silver have energy essentially of the order of 20 keV, they are completely absorbed by the molybdenum foil. However, for photons having a higher energy (approximately between 30 and 35 keV), the probability of absorption by the molybdenum is weaker, of the order of 20 to 25% of that at the K-edge (label 30). Consequently, the Te K_{α} peak, although still present (label 18′), is reduced, whereas the other peaks 16′, 20′ are essentially unaffected compared to peaks 16 and 20.

Returning now to FIG. 2, it is assessed that the two molybdenum peaks (labeled 26 and 28) contribute to about 40% of the total photon emission by the seed wrapped in a molybdenum foil.

Therefore, in this experiment, it is shown that the photon energy spectrum of a iodine-125 seed can be significantly modified by means of a 50 μm thick molybdenum foil wrapping the seed. About 40% of the spectrum modified in such a fashion are comprised of low energy photons, which are well known to be biologically more effective photons.

It is to be noted that when used for the intermediate fluorescent foil, molybdenum also acts as a radio-opaque material, so that additional radio-opaque material such as silver or gold may be omitted.

Considering the above, new seed designs are built. Exemplary implants fabricated in accordance with preferred embodiments of the present invention will now be described with reference to FIGS. 4 to 6.

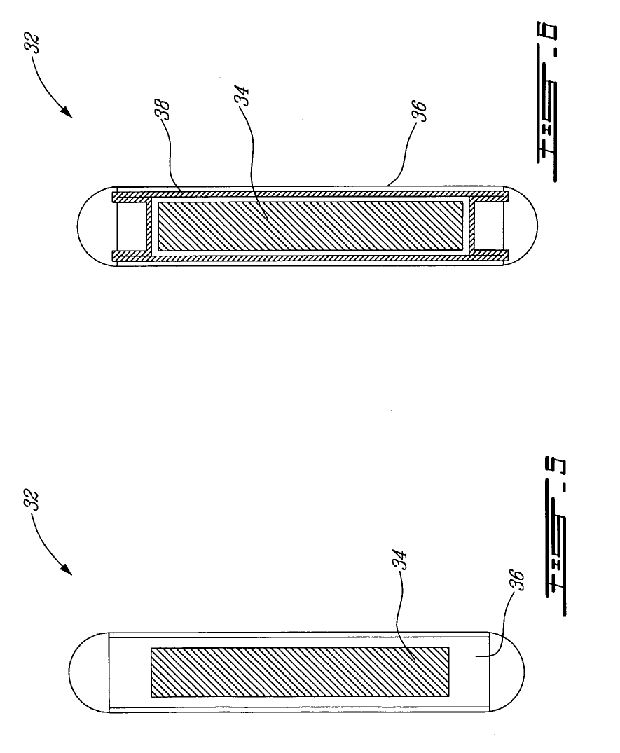

FIGS. 4 and 5 show longitudinal cross-sectional views of brachytherapy source 32, of a generally cylindrical shape, according to specific embodiments of the prior art.

In FIG. 4, the inner core 34 is shown as two small substrates, separated by a radio-opaque rod 33, which immobilize a radioisotope. Currently, this type of implant source includes preferentially paladium-103.

In FIG. 5, the central rod 34 is made of silver, which can act both as a radio-opaque marker and as a substrate for the radioactive isotope. Currently, this type of implant usually includes iodine-125 adsorbed on the silver rod. Alternatively, the silver rod may be replaced by an iridium rod containing at least some iridium-192.

As mentioned previously, in some cases, it is desirable that the radioactive source be encapsulated into a shell 36 preferably made of a biocompatible material, namely a metal such as titanium, for instance.

In comparison, FIG. 6 shows a longitudinal cross-sectional view of a radioactive implant 32 according to a specific embodiment of the present invention. FIG. 6 shows an improved version of the embodiments shown in FIG. 5. The improvement of the present invention can be applied to a plurality of other embodiments comprising that of FIG. 4.

The brachytherapy source 32 is essentially cylindrical in shape, and comprises a central rod 34; an outer shell 36; and an intermediate layer 38 located between the central rod 34 and the outer shell 36.

Similarly to the example illustrated in FIGS. 4 and 5, the central rod 34 is meant to be carrying the radioactive isotope, preferably iodine-125. Alternatively, the radioactive isotope Iridium-192 for instance, has a half-life close to that of iodine-125 (74 days, versus 60 days for iodine-125) and can be used for high dose rates when the central rod 34 is made of iridium containing at least some Iridium-192. The average energy of its X-ray spectrum is significantly higher than that of iodine-125. Nonetheless Iridium-192 could be used instead of iodine-125 provided its X-ray spectrum is shifted to a lower average value by using a fluorescent layer. The same principles would apply: a fluorescent element Z is selected, which can modify the energy spectrum of the radiation before it exits the source depending on the atomic number and on the thickness of the fluorescent layer.

More generally, iodine-125 is not the only possible radioisotope. Other radioactive elements having a suitable half-life can be used, provided their energy spectrum can be modified by a fluorescent layer so as to improve their biological effectiveness.

The outer shell 36 is preferably made of a biocompatible material, namely a metal such as titanium, for instance. Biocompatibility is a desired trait should the source be intended to be left in a patient's body for a relatively long period of time. The thickness of the outer shell 36, as is known in the art (see U.S. Pat. Nos. 3,351,049 and 4,323,055 for example), should be sufficiently large to ensure mechanical strength of the implant, while sufficiently small to enable the radiation to go therethrough; thus, the preferred thickness varies from 0.025 to 0.125 millimeters.

The intermediate layer 38 is made of a fluorescent material. The fluorescent material may comprise more than one element and/or sub-layer in order to modify the energy spectrum of the radioisotope used in the inner core 34. In the case the radioisotope element used in the radioactive inner core 34 is iodine-125, the preferred fluorescent elements for making the fluorescent layer 38 are zirconium, niobium, molybdenum and ruthenium, which atomic numbers are respectively 40, 41, 42 and 44. Atomic number 43 is disregarded solely for a practical reason, since it is a non-natural radioactive element that is expensive to produce.

The biological effectiveness of such an implant is primarily determined by the properties of the fluorescent layer, since the underlying principal is to shift, by means of fluorescence, the photon energy spectrum to a biologically effective range. In the particular case of iodine-125, the biological effective range is essentially between 12 keV, which is a lower limit for a minimal tissue penetration, and 27 keV, which is the average energy of iodine-125. More generally, and depending on the radioisotope used in the inner core, the biologically effective energy range is situated between 12 keV and 100 keV, which is the highest X-ray energy that can be obtained in practice.

The above mentioned shift in energy spectrum, and hence the gain in biological effectiveness, is closely related to the properties of the fluorescent layer, in particular to the atomic number and to the thickness thereof.

Computer simulations are performed to determine the optimal thickness of the X-ray fluorescent layer for a given radioactive inner core. The objective of such simulation is to determine a range of thickness enabling a satisfying X-ray spectrum while yielding an acceptable dose rate.

It is thus found that a inner core made of iodine-125 of about 400 μm can be used as a radioactive element, surrounded by a layer of one of the fluorescent elements mentioned hereinabove.

Alternatively, or in complement, as discussed hereinabove, iridium-192 can be used as a radioactive element. In the case of a radioactive core containing Ir-192, the preferred fluorescent element for making the fluorescent layer is an element with atomic number equal to, or larger than, 39, but not higher than 75. For practical reasons (the element must be solid at room temperature, not too reactive, not too toxic and not an artificial radioactive element), some of the elements in this range are discarded, and the preferred fluorescent elements for making the fluorescent layer around a radioactive core containing Ir-192 are the elements of atomic numbers 39-42, 44-53, 56, 62, 65, 66, 68, 72-75. Again, the thickness is a determinant parameter and the principles that have been applied to a iodine-125 inner core surrounded with an element Z comprised between 40 and 44 apply.

Interestingly, other isotopes of intermediate energy spectrum, situated essentially between those of iodine-125 and irridium-192, such as Ytterbium-169 and samarium-145 for example, and having acceptable half-life (respectively 32 days and 340 days), may be considered as radioactive elements for the inner core.

TABLE 1 shows thickness yielding optimum X-ray emission in the vicinity of 20 keV, resulting essentially in an optimum biological effectiveness, according to the fluorescent element used in the fluorescent layer, in the case of an inner core containing iodine-125. These results have been obtained by means of computer simulations using a software called “GEANT4”. GEANT 4 is a sophisticated particle transport computer code developed and maintained by CERN, the European laboratory for particle physics. Particle-matter interactions such as bremsstrahlung, Compton and photo-electric effect as well as X-ray fluorescence are simulated in a stochastic manner according to cross-sections obtained by mathematical models or as tabulated data.

It is clear that a fluorescent layer made of an element having a smaller atomic number should be thicker than the same made of an element having a higher atomic number. As a result, the thickness of the fluorescent layer is advantageously comprised between 0.020 and 0.300 millimeters.

It is to be noted that there are no restrictions on the diameter of the central rod 34. The size of the central rod 34 may be chosen based on practical considerations, considering that, in order to accommodate existing implantation apparatus, the outer diameter of the brachytherapy source should be preferably within the range between 0.5 and 1.5 millimeters and its overall length should preferably be between 3 and 15 millimeters.

As an example of such a brachytherapy source according to the present disclosure, a brachytherapy source comprising an inner core containing 1 mCi iodine-125 encapsulated in a titanium outer shell, would have the following advantageous characteristics: an inner core having an outer diameter of 0.4 millimeter, a length of 3.0 millimeters; an outer shell made of titanium, having a wall thickness of 0.050 millimeter and an outer diameter of 0.8 millimeter; an intermediate fluorescent layer made of molybdenum consisting in a 0.6-millimeter outer diameter hollow cylinder inserted in the open-ended titanium shell. A 0.45-mm outer diameter cap with a lip to give it a 0.65-mm diameter in its widest portion, a total height of 0.2 mm and a wall thickness of 0.05 millimeter closes each end of the intermediate fluorescent layer.

By way of example, such a layer of molybdenum can be obtained by pressing a 3.6 millimeters long, 1.6 millimeters wide and 0.050 millimeters thick molybdenum foil against a metal rod so as to shape it. The caps for the two ends of the molybdenum layer can be obtained by conventional metal forming techniques, like drawing for instance, using a 0.05 millimeter thick molybdenum foil. The brachytherapy source is assembled and the titanium outer shell is hermetically sealed by various techniques used in the art (see for instance U.S. Pat. No. 4,322,055), such as laser welding, electron beam or tungsten inert gas welding, for instance.

As hereinabove set forth, the present invention provides new designs for radioactive sources that are meant for implantation in the human body for purposes of brachytherapy. The new designs enable increased biological effectiveness relative to similar commercially available sources (other than Pd-103), by providing an improved X-ray spectrum in the vicinity of 20 keV. The present invention thus provides a method to lower the photon energy spectrum of a radiation source, which has a suitable half-life but which energy spectrum needs to be improved in order to yield a higher biological effectiveness.

Although the present invention has been described hereinabove by way of preferred embodiments thereof, it can be modified, without departing from the spirit and nature of the subject invention as defined in the appended claims.

TABLE 1
Thickness and material relation for optimum effectiveness

| Thickness | Fluorescent |
|---|---|
| (Millimeters) | material |
| 0.020 | ruthenium or molybdenum |
| 0.030-0.060 | molybdenum |
| 0.070 | molybdenum or niobium |
| 0.080-0.100 | niobium |
| 0.110-0.140 | niobium or zirconium |
| 0.150-0.300 | zirconium |

What is claimed is:

1. A radioactive source capable of radiating energy in a tissue wherein it is to be implanted, comprising:
a radioactive inner core having an outer surface; and
a layer of fluorescent material disposed around said outer surface;
wherein said central radioactive inner core comprises at least one radioisotope element and said layer of fluorescent material is made of at least one sub-layer of fluorescent material that has a photon energy lower than the energy of said radioisotope element, so that a substantial portion of the energy of the radioisotope element is shifted within a range that is-effective for preventing cell proliferation of said tissue.

2. A radioactive source according to claim 1, wherein said range comprises a lower energy limit of about 12 keV.

3. A radioactive source according to claim 2, wherein said range comprises a higher energy limit of about 100 keV.

4. A radioactive source according to claim 1, wherein said radioisotope is iodine-125.

5. A radioactive source according to claim 1, wherein said radioactive inner core comprises a substrate, at least a portion of said substrate carrying the radioisotope element.

6. A radioactive source according to claim 5, wherein said substrate is cylindrical or spherical.

7. A radioactive source according to claim 5, wherein said substrate has a hollow cylindrical shape.

8. A radioactive source according to claims 5, 6 or 7, wherein said substrate and said source are cylindrical.

9. A radioactive source according to claim 4, wherein said at least one fluorescent layer is made of a material having an atomic number selected in the group comprising the atomic numbers 39, 40, 41, 42, 44, and 45.

10. A radioactive source according to claim 4, wherein said at least one fluorescent layer comprises an element selected from the group consisting of niobium, zirconium, molybdenum, and any combination thereof.

11. A radioactive source capable of radiating energy, comprising:
a radioactive central, substantially cylindrical core, comprising iodine-125 and having an outer surface; and
a layer of fluorescent material comprising at least one element selected from niobium, zirconium and molybdenum, and disposed around said outer surface.

12. A radioactive source according to claim 10 or 11, wherein said at least one fluorescent layer has a thickness essentially within the range between 20 and 300 microns.

13. A radioactive source according to any one of claims 1 to 12, wherein said radioactive source further comprises an outer shell, said outer shell encapsulating said radioactive source and said layer of fluorescent material being located between said central radioactive inner core and said outer shell.

14. A radioactive source according to any one of claims 1 to 12, wherein said radioactive source further comprises an outer shell, said outer shell having an inner surface, said outer shell encapsulating said radioactive source and said layer of fluorescent material being adsorbed on said inner surface of said outer shell.

15. A radioactive source according to claim 13, wherein said outer shell is made of a biocompatible material.

16. A radioactive source according to claim 14, wherein said outer shell is made of a material selected from the group consisting of titanium, titanium alloy, stainless steel, gold and platinum.

17. A radioactive source according to claim 1, wherein said radioactive implant comprises a radio-opaque material.

18. A radioactive source according to claim 17, wherein said radio-opaque material is located in the inner core

19. A radioactive source according to claim 17, wherein said radio-opaque material is silver.

20. A method for fabricating a radioactive source to be implanted in a recipient's tissue, which combines the half-life of a first radioactive element and the energy spectrum of a second radioactive element that has a shorter half-life, comprising the step of shifting down the energy spectrum of the first element towards the energy spectrum of the second radioactive element by placing at least one layer of fluorescent material between the first radioactive element and the tissue to be irradiated.
